(19) United States
(12) Patent Application Publication
(10) Pub. No.: US 2004/0053208 A1
(43) Pub. Date: Mar. 18, 2004

(54) Methods to selectively inactivate parasites in biological compositions

(75) Inventors: Boris Zavizion, Chestnut Hill, MA (US); Andrei Purmal, Waltham, MA (US); Diana Serebryanik, Newton, MA (US)

Correspondence Address: John R. Van Amsterdam, Wolf, Greenfield & Sacks, P.C., 600 Atlantic Avenue, Boston, MA, 02210, US

(73) Assignee: V. I. Technologies, Inc., Watertown, MA (US)

(21) Appl. No.: 10/608,820

(22) Filed: Jun. 27, 2003

Related U.S. Application Data
(63) Continuation-in-part of application No. 09/877,838, filed on Jun. 8, 2001.
Application No. 09/877,838 is a continuation of application No. 09/161,030, filed on Sep. 25, 1998, now abandoned.
Application No. 09/161,030 is a continuation-in-part of application No. 08/855,378, filed on May 13, 1997, now Pat. No. 6,136,586.
Application No. 08/855,378 is a continuation-in-part of application No. 08/705,045, filed on Aug. 29, 1996, now abandoned.
Application No. 08/705,045 is a continuation-in-part of application No. 08/521,245, filed on Aug. 29, 1995, now Pat. No. 6,114,108.

(51) Int. Cl. A01N 1/02; A61K 31/396

(52) U.S. Cl. 435/2; 514/183

(57) ABSTRACT

The invention features a method for inactivating parasites in biological compositions. The method includes contacting the biological composition with an aziridino compound under parasite inactivating conditions.

### RELATED APPLICATIONS

This application is a continuation-in-part of U.S. Ser. No. 09/877,838, filed Jun. 8, 2001, which is a continuation of U.S. Ser. No. 09/161,030 (now abandoned), which is a continuation-in-part of U.S. Ser. No. 08/855,378 (now U.S. Pat. No. 6,136,586), which is a continuation-in-part of U.S. Ser. No. 08/705,045 (now abandoned) which is a continuation-in-part of U.S. Ser. No. 08/521,245 (now U.S. Pat. No. 6,114,108), each of which is hereby incorporated by reference.

### FIELD OF THE INVENTION

This invention relates to methods and compositions for the selective inactivation of parasites and other parasites in biological compositions.

### BACKGROUND OF THE INVENTION

Following traumatic injury (or during surgery), an organism may require a blood transfusion to prevent death due to blood loss. In humans and certain domesticated animals, blood transfusion has enabled the survival of injured individuals who would otherwise have died from blood loss.

Whole blood is composed of many different types of proteins and cells. Blood proteins include antibodies, complement proteins, and proteins involved in the blood clotting cascade. In addition, each of the different types of blood cells plays a unique role in maintaining the health of the organism. Red blood cells, for instance, are essential for the transport of oxygen and carbon dioxide gases to and from the cells of a multicellular organism. Another type of blood cell, a platelet, is involved in initiating blood clotting; thrombocytopenia patients have a platelet deficiency and are prone to bleeding disorders.

One caveat in using blood transfusions is the danger of transmitting blood-borne parasites from donor blood to a recipient. The transmission of parasitic diseases by blood or blood products is a significant problem in medicine. For example, the protozoan parasite Trypanosoma cruzi is the causative agent of Chagas' disease, a chronic debilitating condition affecting millions of people in the Americas. In rural areas of South and Central America and of Mexico, the disease is largely transmitted to man by Reduviid bugs. However, in urban areas of South, Central and North America, Chagas' disease is mostly transmitted by blood transfusion (Transfus Med Rev 13:227, 1999). Screening donor blood for parasites can help reduce the transmission of parasites to recipients, but many screening methods are directed to only a few discrete parasites and are therefore incomplete or less than 100% sensitive. Therefore, there is a need for methods of treatment of biological compositions that would inactivate parasites and make the biological compositions safe for further use, such as transfusion.

### SUMMARY OF THE INVENTION

It has now been discovered that aziridino compounds can be used to inactivate parasites in biological compositions. Accordingly, improved methods and products for the treatment of biological compositions, particularly red blood cells or plasma, are provided according to the invention. In addition, methods are provided for removing the aziridino compound from the biological composition by washing with an inert solution, or by inactivating the aziridino compound by a quenching agent.

According to a first aspect of the invention, a method is provided for selectively inactivating a parasite in a biological composition, comprising contacting the biological composition with a solution comprising an aziridino compound in an amount and under conditions effective to inactivate parasites.

In one embodiment, the biological composition is selected from blood, a red blood cell comprising composition, a red blood cell concentrate, a platelet concentrate, blood plasma, a platelet-rich plasma, a placental extract, a cell culture product or culture medium, a product of fermentation, ascites fluid, serum, a blood cell protein, a blood plasma concentrate, a blood plasma protein fraction, a purified or partially purified blood protein or other component, a supernatant or a precipitate from any fractionation of the plasma, a purified or partially purified blood component (e.g., proteins or lipids), colostrum, milk, urine, saliva, a cell lysate, cryoprecipitate, cryosupernatant, or portion or derivative thereof, compositions containing proteins induced in blood cells, and a composition containing products produced in cell culture by normal or transformed cells. In a related embodiment, the biological composition comprises red blood cells. In another embodiment, the biological composition comprises platelets. The biological composition may alternatively comprise blood plasma. In still another embodiment, the biological composition comprises whole blood.

In some embodiments, the biological composition is derived from humans.

In one embodiment, the aziridino compound contains a linear alkyl group. In another embodiment, the aziridino compound has the structure of formula II:

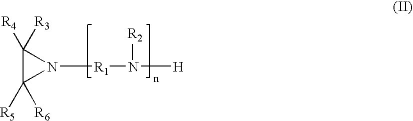

wherein each R₁ is a divalent hydrocarbon moiety containing between two and four carbon atoms, inclusive; each of R₂, R₃, R₄, R₅, and R₆ is, independently, H or a monovalent hydrocarbon moiety containing between one and four carbon atoms, inclusive; and n is an integer between one and ten, inclusive. In a related embodiment, R₂, R₃, R₄, R₅, and R₆ are H.

In another embodiment, the aziridino compound has the structure of formula III:

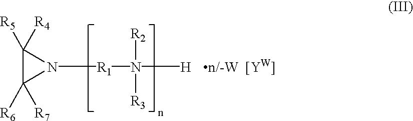

wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; Y is pharmaceutically acceptable counter anion; W is the valency of Y; and n is an integer between one and ten, inclusive. In a related embodiment, R₂, R₃, R₄, R₅, and R₆ are H.

In yet another embodiment, the aziridino compound is an ethyleneimine oligomer. In a related embodiment, the ethyleneimine oligomer is an ethyleneimine dimer. In another embodiment, the ethyleneimine oligomer is an ethyleneimine trimer. The ethyleneimine oligomer may be present at a concentration of at least about 0.005% (vol./vol.), but is not so limited.

In one embodiment, at least 90% of the parasitic pathogens in the biological composition are inactivated. In another embodiment, at least 98% of the parasitic pathogens in the biological composition are inactivated.

In some embodiments, the parasite is selected from the group consisting of Plasmodium, Babesia microti, Babesia divergens, Leishmania tropica, Leishmania, Leishmania braziliensis, Leishmania donovani, Trypanosoma gambiense, Trypanosoma rhodesiense, Trypanosoma cruzi, and Toxoplasma gondii.

In still another embodiment, the method further comprises contacting the biological composition with a parasiticide.

According to a second aspect of the invention, a method is provided for transfusing a subject with a blood product comprising inactivating parasites in a blood product according to any of the methods recited above, and transfusing a subject with the inactivated blood product. In some embodiments, at least some of the aziridino compound is removed prior to transfusion. In a related embodiment, the solution comprising the aziridino compound is removed by washing the biological composition.

In another embodiment, the method further comprises quenching the aziridino compound with a quenching agent. The quenching agent may be soluble, but it is not so limited.

In another embodiment, the transfusion into the mammal is heterologous.

In one embodiment, the subject is a mammal, which may be but is not limited to a human.

In still another embodiment, the method further comprises contacting a biological composition comprising red blood cells with a solution comprising pyruvate, inosine, adenine and phosphate. In a related embodiment, the aziridino compound, pyruvate, inosine, adenine and phosphate increase the levels of 2,3 DPG, ATP or p50 in the contacted red blood cells by at least 25% in comparison to the levels of 2,3 DPG, ATP or p50 in red blood cells not contacted by the aziridino compound, pyruvate, inosine, adenine and phosphate.

According to a third aspect of the invention, a transfusion product is provided comprising a container containing a biological composition in which parasites are inactivated by any of the foregoing methods.

According to a fourth aspect of the invention, a kit is provided for performing the aforementioned methods, comprising one or more containers containing an amount of an aziridino compound effective to inactivate parasites. In one embodiment, the kit further comprises one or more containers containing a parasiticide. In another embodiment, the kit further comprises one or more containers containing a cell washing and/or storage solution.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a bar graph showing the aziridino compound inactivation of T. cruzi (Silvio strain) in heat-inactivated human plasma (23° C.—3 hrs)

FIG. 2 is a bar graph showing dose-dependent inactivation of T. cruzi (Tulahuen strain) in heat-inactivated plasma (23° C.—3 hrs)

FIG. 3 is a graph showing the dose-dependent independent inactivation of the complete cycle of T. cruzi infection (Silvio strain).

FIG. 4 is a bar graph showing the dose-dependent inactivation of T. cruzi (Silvio strain) in leukoreduced CPD/AS-1 RBCC (1 hour—23° C.).

FIG. 5 is a graph showing the kinetics of T. cruzi (Silvio strain) inactivation in CPD/AS-1 RBCC.

FIG. 6 is a graph showing the parasitemia of C3H mice inoculated with aziridino compound treated (0.1%—3 hrs) and non-treated T. cruzi (Tulahuen strain) infected blood.

It is to be understood that the Figures are not required for enablement of the invention.

### DETAILED DESCRIPTION OF THE INVENTION

It has now been discovered that aziridino compounds, including ethyleneimine oligomer compounds, can be used to inactivate parasites in biological compositions such as red blood cell, platelet or plasma solutions. The invention relates to methods and products for the inactivation of parasites in biological compositions including biological compositions used in transfusion.

The methods and products for the inactivation of parasites are based on compounds utilizing aziridino chemistry. The aziridino compounds include in certain embodiments aziridino compounds with an alkyl chain, such as ethyleneimine oligomers, which are positively charged electrophilic molecules chemically related to binary ethyleneimine that has selective reactivity with nucleic acids. As used herein, an “ethyleneimine oligomer” can refer to an ethyleneimine dimer, an ethyleneimine trimer, an ethyleneimine tetramer or a derivative thereof. Methods for synthesis of aziridino compounds, particularly ethyleneimine oligomers, are provided, for example, in U.S. Pat. No. 6,215,003, U.S. Pat. No. 6,559,321 and Kostyanovskii et al., “Oligomer of Aziridines and N-β-Aziridinoethylamides,” Institute of Chemical Physics of the Academy of Sciences of the U.S.S.R. Moscow. Izvestiya Akademii Nauk SSSR, Seriya Khimicheskaya 11:2566-2575 (1988).

The aziridino compounds have a method of action that includes disruption of nucleic acid replication and/or transcription to achieve desirable biological effects. The electrostatic binding of positively charged aziridino compounds such as ethyleneimine oligomers to nucleic acid molecules results in a covalent interaction of the aziridino group with nucleophilic groups of DNA or RNA, predominantly the N-7 position of guanine. Covalent modification of nucleotide bases can cause loss of the base, i.e., formation of abasic sites, or even strand breaks. Abasic sites and strand breaks produced by ethyleneimine oligomer—nucleic acid adducts act as potent stop signals for nucleic acid polymerases. Accordingly, the modified nucleic acids can not serve as templates for replication or transcription. As a result, the aziridino compounds preferably are used for selectively inactivating parasites in biological compositions that include enucleated cells or are free of cells.

Aziridino compounds useful in the methods and composition of the invention preferably contain a moiety having the formula (I):

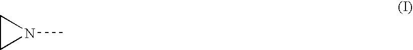

In this three-membered ring, the two carbons are preferably unsubstituted (i.e., they contain hydrogens), but they can be substituted with aliphatic or aromatic hydrocarbon moieties, each containing between one and four carbon atoms, inclusive.

Various aziridino compounds are disclosed in U.S. Pat. No. 6,093,564, and in International Application Number PCT/US02/35501, filed on Nov. 3, 2002, entitled Methods and Compositions for the Modification of Nucleic Acids, the entire disclosures of which are incorporated by reference. The use of these compounds in the methods of the invention is provided herein.

In one set of embodiments, the aziridino compound has the formula (II):

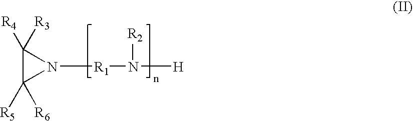

wherein each R₁ is a divalent hydrocarbon moiety containing between two and four carbon atoms, inclusive; each of R₂, R₃, R₄, R₅, and R₆ is, independently, H or a monovalent hydrocarbon moiety containing between one and four carbon atoms, inclusive; and n is an integer between one and ten, inclusive.

In various preferred embodiments, each R₁ contains two or three carbon atoms; each of R₂, R₃, R₄, R₅, and R₆ is H; and n is one or two. For example, ethyleneimine tetramer fits formula (II) when R₁ contains two carbon atoms, and each of R₂, R₃, R₄, R₅, and R₆ is H, and n is three. Similarly, ethyleneimine trimer fits formula (II) where R₁ contains two carbon atoms, each of R₂, R₃, R₄, R₅, and R₆ is H, and n is two, and ethyleneimine dimer fits formula (II) when R₁ contains two carbon atoms, and each of R₂, R₃, R₄, R₅, and R₆ is H, and n is one.

In another set of examples, the compound has the formula (III):

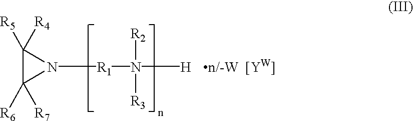

wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; Y is pharmaceutically acceptable counter anion; W is the valency of Y; and n is an integer between one and ten, inclusive.

Aziridino compounds also include open-ring counterparts to the compounds of formula (I). In one example, aziridino compounds useful in the methods of the invention have the formula (IV):

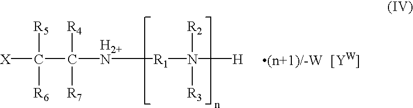

wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; X is Cl or Br; Y is a pharmaceutically acceptable counter anion; W is the valency of Y; and n is an integer between one and ten, inclusive.

In various preferred embodiments of compounds satisfying formula (III) or formula (IV), each R₁ contains two or three carbon atoms; each of R₂, R₃, R₄, R₅, and R₆ is H; and n is one or two. Suitable counter anions include nitrate, sulfate, halide (fluorine, chlorine, bromine, iodine), phosphate, and tosylate ions.

In an additional set of embodiments, the aziridino compound has the formula (V):

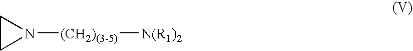

or a salt thereof, wherein each R₁ is, independently, selected from the group consisting of H, C₁₋₄ alkyl, C₂₋₄ alkenyl, phenyl, and benzyl. In particular embodiments, the compound is 1-aziridinepropanamine or 1-aziridinebutanamine (compounds 1 and 2, respectively):

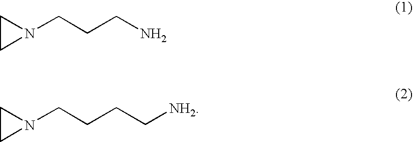

In another additional set of embodiments, the aziridino compound has the formula (VI):

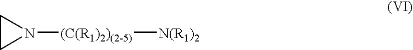

or a salt thereof, wherein each R₁ is, independently, selected from the group consisting of H, C₁₋₄ alkyl, C₂₋₄ alkenyl, phenyl, and benzyl, provided that at least one R₁ is phenyl or benzyl.

Exemplary aziridino compounds that fall within formula (VI) are 3-phenyl-1-aziridinepropanamine, N,N-dibenzyl-1-aziridineethanamine, and N-benzyl-N-ethyl-1-aziridineethanamine, and 2-benzyl-1-aziridineethanamine (compounds 3, 4, 5, and 6, respectively).

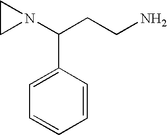

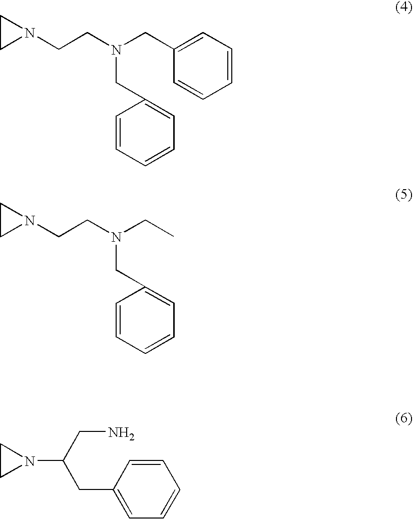

In a further set of embodiments, the aziridino compound has the formula (VII):

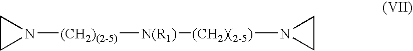

or a salt thereof, wherein R₁ is selected from the group consisting of H, C₁₋₄ alkyl, C₂₋₄ alkenyl, phenyl, and benzyl.

Exemplary compounds that satisfy formula (VII) are 1,1′-[iminobis(dimethylene)]bis aziridine and 1,1′-[iminobis(trimethylene)]bis aziridine (compounds 7 and 8 respectively).

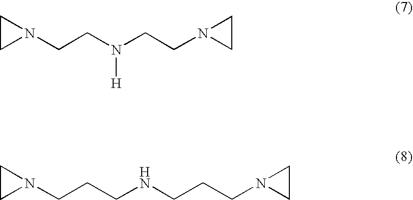

In an additional set of embodiments, the aziridino compound has the formula:

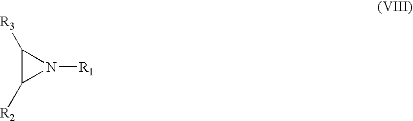

or a salt thereof, wherein R₁ is a C₁₋₄ alkyl and R₂ and R₃ is each, independently, H or a C₁₋₄ alkyl. An exemplary compound of formula (VIII) is:

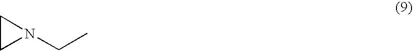

In other embodiments, the aziridino compound is one of the following compounds:

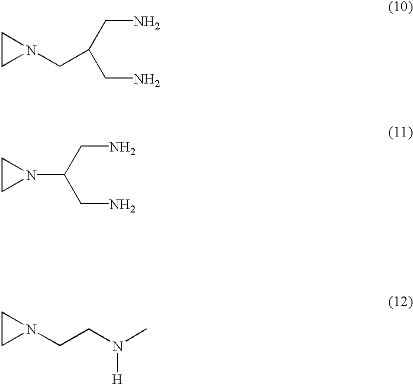

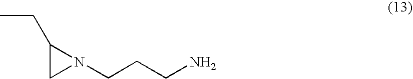

or a salt thereof.

In still another set of embodiments, the aziridino compound has the formula (IX):

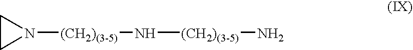

or a salt thereof. An exemplary compound of formula (IX) is:

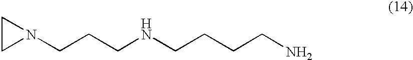

The aziridino ring of the compounds of the invention can be substituted with a structure X—CH₂—CH₂—N—, wherein X is —Cl, —Br, —F, —I, —O—S(═O)₂—CH₃, —O—S(═O)₂—CH₂—C₆H₅, or —O—S(═O)₂—C₆H₄—CH₃. For example, the substituted forms of compounds of formula (V) have the following formula (X):

X—CH₂—CH₂—N—(CH₂)₍₃₋₅₎—N(R₁)₂ (X)

wherein X is —Cl, —Br, —F, —I, —O—S(═O)₂—CH₃, —O—S(═O)₂—CH₂—C₆H₅, or —O—S(═O)₂—C₆H₄—CH₃, each R₁ is, independently, selected from the group consisting of H, C₂₋₄ alkenyl, phenyl, and benzyl.

The aziridino compounds of the present invention are protonated (i.e., positively charged) on one or more nitrogen at physiological pH. For example, protonated compounds of formula (V) (VI), and (VII) have the following respective formulas:

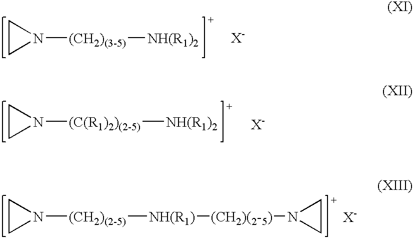

wherein each R₁ is, independently, selected from the group consisting of H, C₂₋₄ alkenyl, phenyl, and benzyl, and X is a pharmaceutically acceptable counter-ion (e.g., sulfate, nitrate, halide, tosylate, phosphate, and the like). For compounds within formula (XII) or (XIII), R₁ can also be C₁₋₄ alkyl. Compounds falling within formula (XII) also have at least one R₁ that is phenyl or benzyl.

These protonated forms of the compounds, described herein, (also referred to as “salts”), and their use in the methods of the invention, are specifically included as being part of the invention.

The compounds useful in the invention described herein also include isomers such as diastereomers and enantiomers, mixtures of isomers, including racemic mixtures, solvates, and polymorphs thereof.

The aziridino compound can be combined prior to, or after, addition of each ingredient to the biological composition. If desired, the aziridino compound can be removed after treating the biological composition. Methods for removing include washing (such as centrifugation-based washing) or solid phase based absorbent removal. Alternatively the aziridino compound can be inactivated by a quenching agent.

As used herein, the term “prevent”, “prevented”, or “preventing” and “treat”, “treated” or “treating” when used with respect to the prevention or treatment of an infectious disease refers to a prophylactic treatment which increases the resistance of a biological solution to a parasite or, in other words, decreases the likelihood that a subject will develop an infectious disease to a parasite following a transfusion of red blood cells treated with the solution containing aziridino compound.

As used herein, a “subject” shall mean a human, a vertebrate mammal including but not limited to a dog, cat, horse, cow, pig, sheep, goat, or non-human primate, e.g., monkey, or a fowl, e.g., chicken. Included within the scope of the present invention are all animals which are susceptible to infectious diseases caused by parasites.

For example, a subject at risk of infectious disease is one for whom the exposure to a parasite or expected exposure to a parasite is known or suspected. A “subject at risk” of developing an infectious disease as used herein is a subject who has any risk of exposure to a parasite following transfusion of a biological solution, e.g. someone who is receiving a transfusion of red blood cells.

An “infectious disease” as used herein, refers to a disorder arising from the invasion of a host, superficially, locally, or systemically, by parasites. Parasites are organisms which depend upon other organisms in order to survive and thus must enter, or infect, another organism to continue their life cycle. The infected organism, i.e., the host, provides both nutrition and habitat to the parasite. The term “parasite” as used herein refers to protozoa, helminths, and ectoparasitic arthropods (e.g., ticks, mites, etc.). Protozoa are single celled organisms which can replicate both intracellularly and extracellularly, particularly in the blood, intestinal tract or the extracellular matrix of tissues. Helminths are multicellular organisms which almost always are extracellular (the exception being Trichinella). Helminths normally require exit from a primary host and transmission into a secondary host in order to replicate. In contrast to these aforementioned classes, ectoparasitic arthropods form a parasitic relationship with the external surface of the host body.

Parasites can be classified based on whether they are intracellular or extracellular. An “intracellular parasite” as used herein is a parasite whose entire life cycle is intracellular. Examples of human intracellular parasites include Leishmania, Plasmodium, Trypanosoma cruzi, Toxoplasma gondii, Babesia, and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at lest one obligate intracellular stage in their life cycles. This latter category of parasites includes Trypanosoma rhodesiense and Trypanosoma gambiense, Isospora, Cryptosporidium, Eimeria, Neospora, Sarcocystis, and Schistosoma. In one aspect, the invention relates to the prevention and treatment of infection resulting from intracellular parasites and obligate intracellular parasites which have at least in one stage of their life cycle that is intracellular. In some embodiments, the invention is directed to the prevention of infection from obligate intracellular parasites which are predominantly intracellular. An exemplary and non-limiting list of parasites for some aspects of the invention is provided herein.

Blood-borne and/or tissues parasites include Plasmodium, Babesia microti, Babesia divergens, Leishmania tropica, Leishmania, Leishmania braziliensis, Leishmania donovani, Trypanosoma gambiense and Trypanosoma rhodesiense (African sleeping sickness), Trypanosoma cruzi (Chagas' disease), and Toxoplasma gondii.

Typical parasites infecting horses are Gasterophilus; Eimeria leuckarti, Giardia; Tritrichomonas equi; Babesia (RBCs), Theileria equi; Trypanosoma; Klossiella equi; Sarcocystis.

Typical parasites infecting swine include Eimeria bebliecki, Eimeria scabra, Isospora suis, Giardia; Balantidium coli, Entamoeba histolytica; Toxoplasma gondii and Sarcocystis, and Trichinella spiralis.

The major parasites of dairy and beef cattle include Eimeria, Cryptosporidium, Giardia; Toxoplasma gondii; Babesia bovis (RBCs), Babesia bigemina (RBCs), Trypanosoma (plasma), Theileria (RBC); Theileria parva (lymphocytes); Tritrichomonas foetus; and Sarcocystis.

Typical parasites infecting sheep and goats include Eimeria, Cryptosporidium, Giardia; Toxoplasma gondii; Babesia (RBC), Trypanosoma (plasma), Theileria (RBC); and Sarcocystis.

Typical parasitic infections in poultry include coccidiosis caused by Eimeria acervulina, E. necatrix, E. tenella, Isospora and Eimeria truncata; histomoniasis, caused by Histomonas meleagridis and Histomonas gallinarum; trichomoniasis caused by Trichomonas gallinae; and hexamitiasis caused by Hexamita meleagridis. Poultry can also be infected Emeria maxima, Emeria meleagridis, Eimeria adenoeides, Eimeria meleagrimitis, Cryptosporidium, Eimeria brunetti, Emeria adenoeides, Leucocytozoon, Plasmodium, Hemoproteus meleagridis, Toxoplasma gondii and Sarcocystis.

Parasitic infections also pose serious problems in laboratory research settings involving animal colonies. Some examples of laboratory animals intended to be treated, or in which parasite infection is sought to be prevented, by the methods of the invention include mice, rats, rabbits, guinea pigs, nonhuman primates, as well as the aforementioned swine and sheep.

Typical parasites in mice include Leishmania, Plasmodium berghei, Plasmodium yoelii, Giardia muris, Hexamita muris; Toxoplasma gondii; Trypanosoma duttoni (plasma); Klossiella muris; Sarcocystis. Typical parasites in rats include Giardia muris, Hexamita muris; Toxoplasma gondii; Trypanosoma lewisi (plasma); Trichinella spiralis; and Sarcocystis. Typical parasites in rabbits include Eimeria; Toxoplasma gondii; Nosema cuniculi; Eimeria stiedae, and Sarcocystis. Typical parasites of the hamster include Trichomonas; Toxoplasma gondii; Trichinella spiralis; and Sarcocystis. Typical parasites in the guinea pig include Balantidium caviae; Toxoplasma gondii; Klossiella caviae; and Sarcocystis.

Parasiticides are agents that kill parasites directly and can be used in combination with the methods and compositions described herein. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Parasiticides used in non-human subjects include piperazine, diethylcarbamazine, thiabendazole, fenbendazole, albendazole, oxfendazole, oxibendazole, febantel, levamisole, pyrantel tartrate, pyrantel pamoate, dichlorvos, ivermectin, doramectic, milbemycin oxime, iprinomectin, moxidectin, N-butyl chloride, toluene, hygromycin B thiacetarsemide sodium, melarsomine, praziquantel, epsiprantel, benzimidazoles such as fenbendazole, albendazole, oxfendazole, clorsulon, albendazole, amprolium; decoquinate, lasalocid, monensin sulfadimethoxine; sulfamethazine, sulfaquinoxaline, metronidazole.

Parasiticides used in horses include mebendazole, oxfendazole, febantel, pyrantel, dichlorvos, trichlorfon, ivermectin, piperazine; for S. westeri: ivermectin, benzimiddazoles such as thiabendazole, cambendazole, oxibendazole and fenbendazole. Useful parasiticides in dogs include milbemycin oxine, ivermectin, pyrantel pamoate and the combination of ivermectin and pyrantel. The treatment of parasites in swine can include the use of levamisole, piperazine, pyrantel, thiabendazole, dichlorvos and fenbendazole. In sheep and goats anthelmintic agents include levamisole or ivermectin. Caparsolate has shown some efficacy in the treatment of D. immitis (heartworm) in cats.

Agents used in the prevention and treatment of protozoal diseases in poultry, particularly trichomoniasis, can be administered in the feed or in the drinking water and include protozoacides such as aminonitrothiazole, dimetridazole (Emtryl), nithiazide (Hepzide) and Enheptin.

The term “effective amount” of an aziridino compound (optionally combined with other non-aziridino compounds as described herein) refers to the amount necessary or sufficient to realize a desired biologic effect. For example, an effective amount of an aziridino compound for preventing infectious disease involving transfusion of a red blood cell solution is that amount necessary to prevent the infection with the parasite, is that amount necessary to decrease the amount of the infection that would otherwise occur in the absence of the aziridino compound. In particular, an effective amount for inactivating parasites in biological compositions is that amount of an aziridino compound that reduces an activity of a parasite, such as infection of cells, replication or transcription of nucleic acids.

In some embodiments of the invention, an aziridino compound and one or more non-aziridino compounds are used in a synergistic amount effective to selectively inactivate parasites and reduce parasitic infection. A synergistic amount is that amount which produces an effect that is greater than the sum of the individual effects of either agent alone. For instance, in some embodiments of the invention, the physiological effect of parasite inactivation is a reduction in the number of cells infected with a parasite (e.g. following transfusion with a blood product). A synergistic amount of the aziridino and non-aziridino compounds is that amount which produces a reduction in infected cells that is greater than the sum of the infected cells reduced by either the aziridino compound or the non-aziridino compound alone. In other embodiments, the physiological result is a reduction in the number of parasites in the body. The synergistic amount in this case is that amount which produces the reduction that is greater than the sum of the reduction produced by either the aziridino compound or the non-aziridino compound alone.

In accordance with the methods of the invention, parasites can be modified by contacting a biological composition with about 0.00001 to about 0.250 M, preferably about 0.0001 to about 0.015 M of an inactivating aziridino compound of the invention. Preferably the aziridino compound is contained in a solution having an ionic strength of about 0.01 M to about 0.5 M and a pH of about 4.5 to 8.5, preferably about 6.0 to 8.0, more preferably about 6.5 to about 7.5. In preferred embodiments, the inactivation reaction is carried out at a temperature of about 4° C. to about 60° C., preferably 4° C. to about 30° C., for a time sufficient to inactivate the parasites in the biological composition (e.g., by modifying parasite nucleic acids) to the desired extent.

The aziridino compounds can be used also on the basis of volume/volume amounts. Preferred volume/volume concentrations include from about 0.0001% to about 0.5% vol./vol. (i.e., 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.010%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%,0.027%, 0.028%, 0.029%, 0.03%, 0.04%, 0.05%, 0.06%, 0.07%, 0.08%, 0.09%, 0.1%, 0.2%, 0.3%, 0.4% and 0.5% and fractional amounts therebetween).

The aziridino compounds are mixed with the biological composition for a desired length of time. The incubation times depend on the concentration of the aziridino compound used, the type of parasite that is being inactivated, the incubation temperature and the biological composition being treated. The reaction time can range from about 1 minute to about 500 hours and can be, for example, about 1 minute, about 10 minutes, about 30 minutes, about 1 hour, about 2 hours, about 3 hours, about 4 hours, about 5 hours, about 6 hours, about 12 hours, about 18 hours, about 24 hours, about forty-eight hours or about one hundred and forty-eight hours. Incubation times for inactivating parasites in various biological compositions can vary. For example, when the biological composition is a red blood cell concentrate, a preferred incubation time is from 1 to 24 hrs at 23° C.

The aziridino compounds may be used per se (neat) or in the form of a pharmaceutically acceptable salt. The salts used can be any of those normally used in biochemical applications, including sodium, potassium, acetate, and so on. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

The practitioner can adjust the pH of the solution using many buffers customarily used in the art to handle biopolymers or cells, such as acetate, HEPES, MOPS, and so forth. Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives, should the use of these be desired in treated compositions, include benzalkonium chloride (0.003-0.03% w/v); chlorobutanol (0.3-0.9% w/v); parabens (0.01-0.25% w/v) and thimerosal (0.004-0.02% w/v).

Therapeutic doses of the non-aziridino compounds for use in combination with the administration (e.g., transfusion) of biological compositions (e.g., blood products) treated with aziridino compounds are well known in the field of medicine for the inactivation of parasites. These dosages have been extensively described in references such as Remington's Pharmaceutical Sciences, 18th ed., 1990; as well as many other medical references relied upon by the medical profession as guidance for the treatment of parasitic infections.

For any compound described herein an effective amount can be initially determined from in vitro assays and/or based on known effective amounts for known agents. For instance, the effective amount of aziridino compounds useful for inactivation of parasites in human plasma can be assessed using standard in vitro assays. These assays can be used to determine an effective amount of the particular aziridino compound. Effective amounts can also be determined from in vivo methods (including animal models) as will be well known to, and routinely performed by, one of ordinary skill in the art. Adjusting the dose to achieve maximal efficacy based on the methods described above and other methods as are well-known in the art is well within the capabilities of the ordinarily skilled artisan. Exemplary methods for determining an effective amount of aziridino compound for parasite inactivation are provided in the Examples below.

“Biological composition” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells.

“Biopolymer” or “biological molecule” refers to any class of organic molecule normally found in living organisms including, for example, nucleic acids, polypeptides, post-translationally modified proteins (e.g., glycoproteins), polysaccharides, and lipids. Biopolymer-containing compositions include, for example, blood cell proteins, blood plasma, a blood plasma fractionation precipitate, a blood plasma fractionation supernatant, cryoprecipitate, cryosupernatant or portion or derivative thereof, serum, or a non-blood product produced from normal or transformed cells (e.g., via recombinant DNA technology).

The invention also includes biological compositions treated by the herein described methods, as well as methods for using the treated biological compositions. For example, blood cells treated using the methods and/or compositions described herein can be transfused (either heterologously or autologously) into a subject. Other biological compositions also can be administered using standard methodologies following parasite inactivation.

“Kits” include one or more vials or containers containing an aziridino compound and can be provided in a single vial or container, if desired. The kits are optionally provided with instructions for using the compositions in the vials.

“Inactivating,” “inactivation,” or “inactivate,” when referring to parasites means diminishing or eliminating the number of infectious parasites measured as a decrease in the infectious titer or number of infectious parasites per volume (e.g. per ml of a treated biological composition). In addition, these terms include reducing or abolishing parasite's whipping movement, parasite's ability to invade host cells and to multiply inside them, parasite's ability to undergo a full cycle of growth and to cause parasitemia following intraperitoneal inoculation. The aforementioned are illustrated in detail in the examples bellow. Preferably the methods of the invention result in at least 50% of the parasites in the treated preparation are inactivated, preferably at least 70% of the parasites are inactivated, more preferably at least 80%, still more preferably at least 90%, still more preferably at least 95%, still more preferably, at least 99%, and most preferably, 100% of the parasites in the treated preparation are inactivated. The number of parasites in a preparation may be measured by the number or titer of infectious parasitic particles per ml of preparation. Such a measurement may be accomplished by a variety of well known parasite titer assays well known to a person of ordinary skill in the art, some of which are used in the Examples below.

When referring to nucleic acids, the terms “inactivating,” “inactivation,” or “inactivate” mean to substantially eliminate the template activity of DNA or RNA, for example, by destroying the ability to replicate nucleic acids, transcribe nucleic acids or translate a nucleic acid message. For example, the inhibition of translation of an RNA molecule can be determined by measuring the amount of protein encoded by a definitive amount of RNA produced in a suitable in vitro or in vivo translation system. Other assays for measuring replication, transcription or translation of nucleic acids are well known to those of skill in the art and include, e.g., PCR, nucleic acid hybridization and ELISA assays.

“Parasite inactivating conditions” refer to the conditions under which the parasitic particles are incubated with the selective inactivating agents of this invention, including, for example, time of treatment, pH, temperature, salt composition, and concentration of selective inactivating agent, so as to inactivate the parasite to the desired extent. Parasite inactivating conditions are selected from the conditions described herein for the selective inactivation of parasites in biological compositions.

By “nucleic acid” is meant both DNA and RNA, both single and double stranded.

By an “enucleated cell” is meant a cell which, when mature, lacks a nucleus. Preferred examples of enucleated cells are platelets and red blood cells.

By a “solution that does not quench an ethyleneimine dimer” is meant a solution that does not contain a quenching agent (e.g., a thiophosphate or a thiosulfate). A quenching agent, when contacted with ethyleneimine dimer, renders the contacted ethyleneimine dimer non-toxic. Preferred solutions that are incapable of reacting with an ethyleneimine dimer are unbuffered saline and water.

By a “quenching agent” is meant a thiophosphate or a thiosulfate, or a compound containing a thiophosphate or a thiosulfate that, when contacted with ethyleneimine dimer, is capable of rendering the contacted ethyleneimine dimer non-toxic.

### EXAMPLES

We have discovered a method to selectively inactivate parasites in biological compositions by treating the composition with an aziridino compound. For example, most mature mammalian red blood cells, unlike those of other vertebrate animals, lack nuclei and, hence, lack nucleic acid. Thus, treatment of the cells with aziridino compound that inactivates nucleic acids allows for the selective inactivation of any parasites contaminating the red blood cell preparation, while leaving the red blood cells unaffected. Likewise, since mature platelet cells (also known as platelets) lack nuclei, they are similarly unaffected by treatment with parasite-inactivating aziridino compound.

The invention also provides a method for removing the aziridino compound from the treated biological composition (e.g., blood), prior to use of the composition by repeatedly washing the composition with a solution that does not quench the aziridino compound (e.g., sterile unbuffered saline) or by solid phase removal of the aziridino compounds. Where the biological composition is a composition containing cells, the treated cells may be washed by repeated steps of resuspension in a solution that does not quench an aziridino compound and isolating the cells by centrifugation. Where the biological composition is a cell-free composition (e.g., milk), the treated milk proteins may be, for example, diluted with a solution that does not quench an aziridino compound, and then dialyzed to remove the aziridino compound.

Since the goal of a blood transfusion is often the transfer of red blood cells, it may be desirable to separate these cells from the other blood components, such as white blood cells (e.g., lymphocytes, neutrophils, and platelets) and biological molecules (e.g., clotting factors and complement).

Standard methods exist for the separation of red blood cells from other blood components. For example, a Ficoll or Percoll gradient may be used to separate the different components of whole blood based on their differences in density. Such gradients may be generated using reagents commercially available from, for example, Amersham Biosciences (Piscataway, N.J.).

In addition, commercially available systems such as the MCS®+ Apheresis System (commercially available form Haemonetics Corp., Braintree, Mass.) may be used to isolate red blood cells from whole blood. It should be noted that this system may also be used to separate other enucleated cells (e.g., platelets) from whole blood.

### Example 1

### Inactivation of Virulent Trypanosoma Cruzi Trypomastigotes by the INACTINE™ Process

Background:

In this study, we evaluated the efficacy of the INACTINE™ technology (aziridino compounds) to inactivate T. cruzi in AS-1 diluted (15%) fresh human plasma as a prelude to determine efficacy in transfusion of blood contaminated with T. cruzi. For red blood cell concentrate (RBCC), the INACTINE™ process includes incubation of red blood cells (RBCs) with 0.1% (v/v) of ethyleneimine oligomer at 23° C. for 24 hours followed by washing by a procedure optimized for the removal of ethyleneimine oligomer to the level of ≦50 ng/mL.

Methods:

T. cruzi parasites were maintained in Vero cells. Two strains of T. cruzi were used in the study: Tulahuen MV-13, which is highly virulent for experimental animals; and Silvio X-10/4, which has more potential to infect cells in vitro. Trypomastigotes were harvested by centrifugation and resuspended in DMEM with 0.1% bovine serum albumin (BSA). The effect of ethyleneimine oligomer on the survival of T. cruzi trypomastigotes was studied in vitro in fresh human heat-inactivated plasma obtained from CPD-collected blood supplemented with AS-1 additive solution, and in CPD/AS-1 RBCC.

Fresh human pooled plasma obtained from CPD-collected whole blood was incubated at 58° C. for 45 min, centrifuged and filter-sterilized. Units of A+ whole blood (≦48 hours post-donating) collected from normal random volunteers into citrate-phosphate-dextrose (CPD) anticoagulant were purchased from the American Red Cross New England Region Blood Services (Dedham, Mass.). The blood was leukoreduced using Pall leukoreduction filters (Pall RCXL1 High Efficiency Leukocyte Removal Filter system). After plasma removal, RBC concentrate was supplemented with AS-1 storage solution (hematocrit values of approximately 50-55%).

For inactivation in plasma, parasites (4 to 8×10⁶ organisms/mL) were treated at 23° C. for various times and different concentrations of PEN110 (ethyleneimine oligomer solution), washed by centrifugation to remove residual drug, and resuspended in DMEM-0.1% BSA solution. T. cruzi treated or not treated with PEN110 (ethyleneimine oligomer) were used to infect Vero cells in 96-well microtiter plates. Infection was allowed to proceed for various numbers of days, and it was ascertained in two ways. Infected monolayers were fixed and stained with DiffQuick (Dade Behring, Deerfield, Ill.) and analyzed under a microscope for infected cells. More than 1500 cells were analyzed for each concentration of PEN110 (ethyleneimine oligomer) or time point. Infection was allowed to proceed through its full cell cycle, which ends with the release of swimming trypomastigotes. Treatment samples were incubated at 23° C. for specified time with various concentrations of ethyleneimine oligomer added to the sample as neutral 20×stock in 0.25M NaH₂PO₄. Control samples were mock-treated with 0.25M sodium phosphate (pH 7.2) and incubated at 23° C. for the same period of time as the treated samples. After the treatment, ethyleneimine oligomer was removed by three cycles of washing with centrifugation (2,000 rpm, 1615×g, 4 min.).

For in vivo inactivation studies the following protocol was followed. Fresh human CPD/AS-1 RBCCs containing T. cruzi parasites (Tulahuen MV-13 strain) were treated with 0.1% ethyleneimine oligomer solution for 3 hours at 23° C. Subsequently, the RBCCs were washed to remove any residual ethyleneimine oligomer, resuspended in AS-3 storage solution, and inoculated intraperitoneally in C3H mice. Each experimental group contained 5 animals. Each animal received approximately 10⁴ parasites. Numbers of parasites in blood (parasitemia) collected from the tail vein were quantified weekly in a Neubauer chamber starting from day 10 after inoculation.

Results:

In vitro inactivation studies. As shown in Table I, treatment of trypomastigotes (Silvio strain) with various concentrations of PEN110 (ethyleneimine oligomer) significantly reduced (at 0.005 and 0.01% concentration) or completely aborted (at 0.05 and 0.1% concentration) parasite whipping movement in the human plasma culture medium.

TABLE I
Effect of PEN110 Ethyleneimine Oligomer Treatment
on T. cruzi (Silvio strain) Motility

| PEN110 (v/v %) | 0 | 0.001 | 0.005 | 0.01 | 0.05 | 0.1 |
|---|---|---|---|---|---|---|
| T. cruzi motility* | ++++ | ++++ | ++ | + | − | − |

*motility was arbitrarily counted as ++++ for parasites from mock-treated control

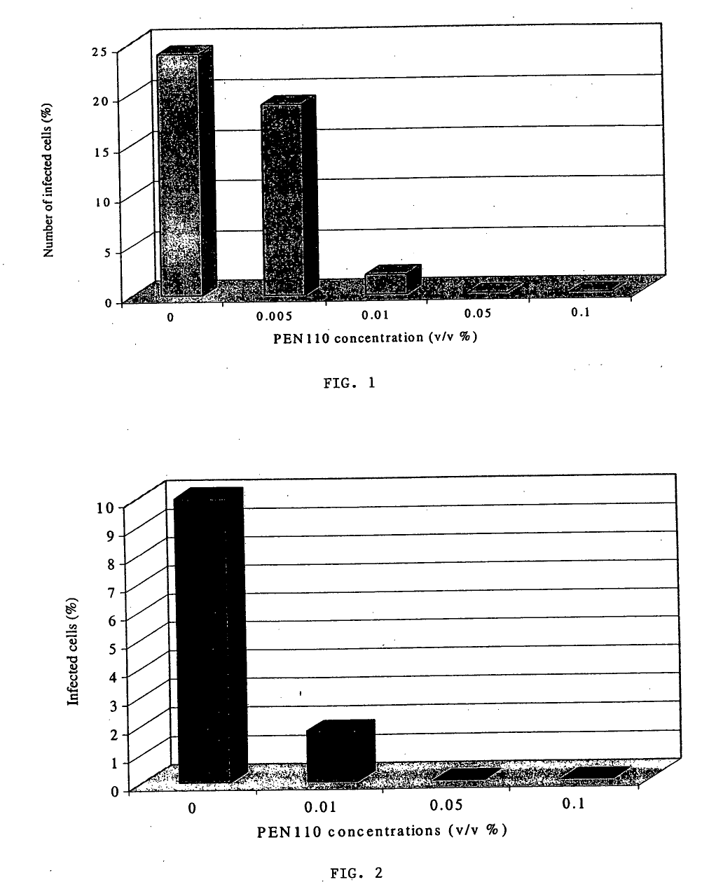

Further investigation revealed that in human plasma, PEN110 (ethyleneimine oligomer) treated T. cruzi (both Silvio and Tulahuen strains) significantly (at 0.005 and 0.01% concentration) or completely (at 0.05 and 0.1% concentration) loses its ability to invade and to multiply in standard host cells, cultured Vero cells (FIGS. 1-2).

The study of the full cycle of the parasite growth in cell culture, which includes parasite entry into the cell, differentiation, multiplication, re-differentiation and exit of newly de-differentiated parasites into the extracellular environment, showed the exquisite sensitivity of T. cruzi to ethyleneimine oligomer treatment since concentrations of 0.01% or above of PEN110 (ethylene oligomer) completely inhibited full cycle of T. cruzi growth (FIG. 3).

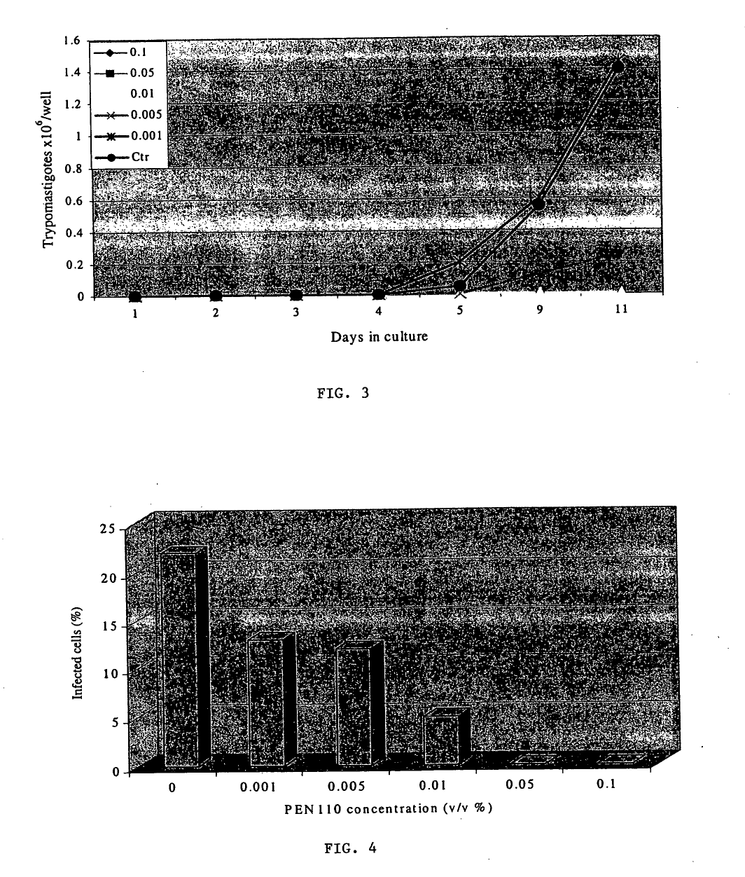

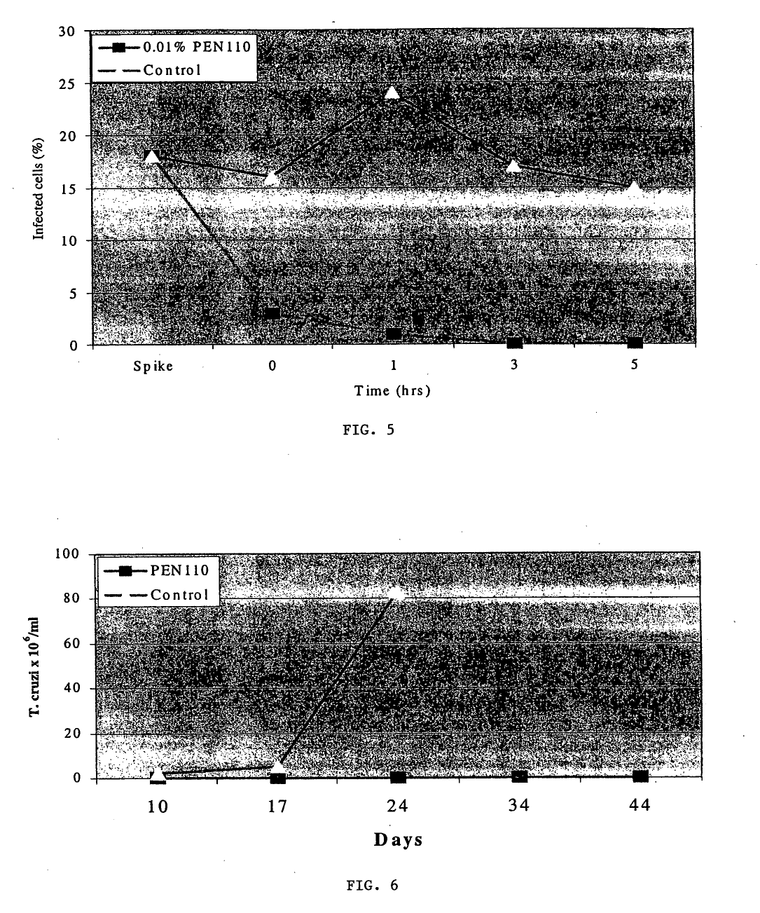

Dose-response and time course experiments demonstrated that PEN110 (ethyleneimine oligomer), upon contact with T. cruzi trypomastigotes in human RBC concentrates at 23° C., is very potent in inhibiting parasite invasion and proliferation in cultured Vero cells. PEN110 (ethyleneimine oligomer) concentrations higher than 0.01% effectively inhibited parasite invasion after less than 3 hours of incubation (FIGS. 4-5).

In vivo inactivation study. Human CPD/AS-1 RBC concentrates were spiked with T. cruzi trypomastigotes, treated with ethyleneimine oligomer or mock-treated (Control sample), washed and inoculated intraperitoneally in C3H mice.

C3H mice infected with control samples exhibited high parasitemia beginning from day 17 after inoculation (FIG. 6) and all died 40 days post-inoculation (Table II).

TABLE II
Survival of C3H Mice Inoculated with PEN110 (0.1%, 3 hrs) Treated
and Non-Treated T. cruzi (Tulahuen strain) Infected Blood

| | |
|---|---|
| Number of | |
| animals | Days post inoculation |

| Treatment | inoculated | 10 | 17 | 24 | 34 | 44 |
|---|---|---|---|---|---|---|
| Sham | 5 | 5 | 5 | 4 | 1 | 0 |
| control | | | | | | |
| PEN110 | 5 | 5 | 5 | 5 | 5 | 5 |
| treatment | | | | | | |

In contrast, mice inoculated with the infected blood treated according to the INACTINE™ process displayed no parasitemia after all course of observation (FIG. 6 and Table II).

Conclusions:

The results show unequivocally that the INACTINE™ technology is very potent in inhibiting T. cruzi trypomastigotes motility and their ability to infect and multiply in cultured cells if PEN110 (ethyleneimine oligomer) is allowed to be in contact with the parasite in human plasma only for a few hours. Human RBCCs did not reduce the effectiveness of PEN110 (ethyleneimine oligomer) to inactivate T. cruzi invasion of cells in culture.

Experiments with the murine model of Chagas' disease demonstrated that treatment of deliberately-contaminated RBC concentrates with the INACTINE™ process prevents the development of transfusion-associated human Chagas' disease.

### Other Embodiments

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

Without further elaboration, it is believed that one skilled in the art can, based on the description herein, utilize the present invention to its fullest extent. All publications and patent applications mentioned in this specification are herein incorporated by reference to the same extent as if each independent publication or patent application was specifically and individually indicated to be incorporated by reference.

What is claimed is:

1. A method for selectively inactivating a parasite in a biological composition, comprising contacting the biological composition with a solution comprising an aziridino compound in an amount and under conditions effective to inactivate parasites.

2. The method of claim 1, wherein the biological composition is selected from blood, a red blood cell comprising composition, a red blood cell concentrate, a platelet concentrate, blood plasma, a platelet-rich plasma, a placental extract, a cell culture product or culture medium, a product of fermentation, ascites fluid, serum, a blood cell protein, a blood plasma concentrate, a blood plasma protein fraction, a purified or partially purified blood protein or other component, a supernatant or a precipitate from any fractionation of the plasma, a purified or partially purified blood component (e.g., proteins or lipids), colostrum, milk, urine, saliva, a cell lysate, cryoprecipitate, cryosupernatant, or portion or derivative thereof, compositions containing proteins induced in blood cells, and a composition containing products produced in cell culture by normal or transformed cells.

3. The method of claim 2, wherein the biological composition comprises red blood cells.

4. The method of claim 2, wherein the biological composition comprises platelets.

5. The method of claim 2, wherein the biological composition comprises blood plasma.

6. The method of claim 2, wherein the biological composition comprises whole blood.

7. The method of claim 2, wherein the biological composition is derived from humans.

8. The method of claim 1, wherein the aziridino compound contains a linear alkyl group.

9. The method of claim 1, wherein the aziridino compound has the structure of formula II: 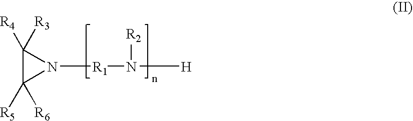
wherein each R₁ is a divalent hydrocarbon moiety containing between two and four carbon atoms, inclusive; each of R₂, R₃, R₄, R₅, and R₆ is, independently, H or a monovalent hydrocarbon moiety containing between one and four carbon atoms, inclusive; and n is an integer between one and ten, inclusive.

10. The method of claim 6, wherein R₂, R₃, R₄, R₅, and R₆ are H.

11. The method of claim 1, wherein the aziridino compound has the structure of formula III: 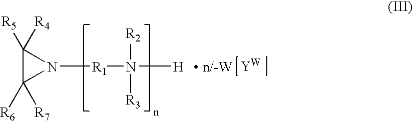
wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; Y is pharmaceutically acceptable counter anion; W is the valency of Y; and n is an integer between one and ten, inclusive.

12. The method of claim 8, wherein R₂, R₃, R₄, R₅, and R₆ are H.

13. The method of claim 1, wherein the aziridino compound is an ethyleneimine oligomer.

14. The method of claim 10, wherein the ethyleneimine oligomer is an ethyleneimine dimer.

15. The method of claim 10, wherein the ethyleneimine oligomer is an ethyleneimine trimer.

16. The method of claim 13, wherein the ethyleneimine oligomer is present at a concentration of at least about 0.005% (vol./vol.).

17. The method of claim 1, wherein at least 90% of the parasitic pathogens in the biological composition are inactivated.

18. The method of claim 17, wherein at least 98% of the parasitic pathogens in the biological composition are inactivated.

19. The method of claim 1, wherein the parasite is selected from the group consisting of Plasmodium, Babesia microti, Babesia divergens, Leishmania tropica, Leishmania, Leishmania braziliensis, Leishmania donovani, Trypanosoma gambiense, Trypanosoma rhodesiense, Trypanosoma cruzi, and Toxoplasma gondii.

20. A method for transfusing a subject with a blood product comprising
inactivating parasites in a blood product according to the method of any of claims 1-19, and
transfusing a subject with the inactivated blood product.

21. The method of claim 20, wherein at least some of the aziridino compound is removed prior to transfusion.

22. The method of claim 21, wherein the solution comprising the aziridino compound is removed by washing the biological composition.

23. The method of claim 20, further comprising quenching the aziridino compound with a quenching agent.

24. The method of claim 23, wherein the quenching agent is soluble.

25. The method of claim 20, wherein the transfusion into the mammal is heterologous.

26. The method of claim 20, wherein the subject is a mammal.

27. The method of claim 26, wherein the mammal is a human.

28. A transfusion product comprising a container containing a biological composition in which parasites are inactivated by the method of any of claims 1-19.

29. The method of claim 20, further comprising contacting a biological composition comprising red blood cells with a solution comprising pyruvate, inosine, adenine and phosphate.

30. The method of claim 29, wherein the aziridino compound, pyruvate, inosine, adenine and phosphate increase the levels of 2,3 DPG, ATP or p50 in the contacted red blood cells by at least 25% in comparison to the levels of 2,3 DPG, ATP or p50 in red blood cells not contacted by the aziridino compound, pyruvate, inosine, adenine and phosphate.

31. The method of claim 1, further comprising contacting the biological composition with parasiticide.

32. A kit for performing the method of claim 1, comprising one or more containers containing an amount of an aziridino compound effective to inactivate parasites.

33. The kit of claim 32, further comprising one or more containers containing a parasiticide.

34. The kit of claim 32, further comprising one or more containers containing a cell washing and/or storage solution.
